(19) United States
(12) Patent Application Publication
(10) Pub. No.: US 2018/0221636 A1
(43) Pub. Date: Aug. 9, 2018

(54) Artificial Pancreas System and Method Therefor

(71) Applicant: These Three Medical Inc., Cutler Bay, FL (US)

(72) Inventors: Marc Theodore Stein, Chandler, AZ (US); Adam Gabriel Stein, Phoenix, AZ (US)

(73) Assignee: These Three Medical Inc., Cutler Bay, FL (US)

(21) Appl. No.: 15/624,658

(22) Filed: Jun. 15, 2017

Related U.S. Application Data
(60) Provisional application No. 62/351,243, filed on Jun. 16, 2016.

(51) Int. Cl. A61M 37/00; A61N 1/05; A61M 5/142; A61M 5/172; A61M 5/145; A61N 1/32

(52) U.S. Cl. CPC A61M 37/0092; A61N 1/0548; A61M 5/14276; A61M 5/1723; A61M 5/14586; A61M 2037/0007; A61M 2230/201; A61M 2205/8206; A61M 2230/50; A61M 2205/3324; A61N 1/325

(57) ABSTRACT

An artificial pancreas system comprising electronic circuitry, a glucose sensor, and an insulin delivery system configured to be placed in the mouth. The artificial pancreas system is configured to measure glucose levels in saliva of the mouth and deliver insulin to the venous system of the mouth. The amount of insulin delivered is a function of the glucose measurement data measured by the glucose sensor. The artificial pancreas system is configured to excite the delivery of insulin ultrasonically and provide electrical stimulation to tissue in the mouth. The artificial pancreas system is configured to form channels in the tissue wherein a portion of the delivered insulin is within the channels in the tissue to be absorbed by the venous system of the mouth.

### FIELD

The present invention pertains generally to the measurement of physical parameters, and particularly to, an insulin delivery system with closed loop feedback glucose monitoring.

### BACKGROUND

A reported 16 million people in the USA alone, and 120 million people world-wide suffer from diabetes. The world-wide number of diabetes sufferers is anticipated to be 300 million by the year 2025. A new case of diabetes is diagnosed every 40 seconds. Diabetes, and specifically the failure to consistently regulate blood glucose concentrations to be within normally tight physiological bounds is directly related to the death of one U.S. citizen every 3 minutes. Almost every major system, organ, and function in the human body is impacted by a lack of glycemic regulation.

Present diabetic therapy protocols comprise multiple injection regimens and external infusion devices that deliver insulin into the peripheral tissue. External, percutaneous, continuous glucose monitors (CGMs) are sometimes used to sample and determine sugar levels in the peripheral tissues and wirelessly report the data to external controllers. Alternatively, testing kits are used to take blood samples. The testing kits are used to measure glucose levels multiple times per day. A dose of insulin based on the blood test kit can be injected into the body to support regulation of the patient glucose levels. It would be of great benefit if a measurement system and delivery system could be provided that better regulates glucose levels.

### BRIEF DESCRIPTION OF THE DRAWINGS

Various features of the system are set forth with particularity in the appended claims. The embodiments herein, can be understood by reference to the following description, taken in conjunction with the accompanying drawings, in which:

FIG. 1 illustrates an artificial pancreas system in accordance with an example embodiment;

FIG. 2 is an illustration of an ingestible capsule of the artificial pancreas system in accordance with an example embodiment;

FIG. 3 is an illustration of the ingestible capsule illustrating electronic circuitry to measure a parameter and form a closed loop that adjusts the parameter based on quantitative measurement data in accordance with an example embodiment;

FIG. 4 is an illustration of the ingestible capsule illustrating coatings that store and release a chemical in accordance with an example embodiment;

FIG. 5 is an illustration of the ingestible capsule passing through the GIT where one or more sensors activate when approaching a predetermined location in accordance with an example embodiment;

FIG. 6 is an illustration of a sensor interface comprising one or more sensors in accordance with an example embodiment;

FIG. 7 is an illustration of the mouth and GIT in accordance with an example embodiment;

FIG. 8 is an illustration of an artificial pancreas system in accordance with an example embodiment;

FIG. 9 is an illustration of a side view of a portion of the artificial pancreas system in accordance with an example embodiment;

FIG. 10 is an illustration of the second side of the printed circuit board in accordance with an example embodiment;

FIG. 11 is a block diagram of the electronic circuitry in accordance with an exemplary embodiment;

FIG. 12 is an illustration of pump 240 in accordance with an exemplary embodiment;

FIG. 13 is an illustration of a timing diagram of the pump in accordance with an example embodiment;

FIG. 14 is an illustration of artificial pancreas system 200 in accordance with an example embodiment;

FIG. 15 is an illustration of an enclosure that protects the artificial pancreas system from an external environment in accordance with an example embodiment;

FIG. 16 is an illustration of the artificial pancreas system protected from an external environment in accordance with an example embodiment;

FIG. 17 is an illustration of a bladder coupling to the pump in accordance with an example embodiment; and

FIG. 18 is an illustration of a second piezo-electric membrane on the first piezo-electric membrane in accordance with an example embodiment.

### DETAILED DESCRIPTION

Embodiments of the invention are broadly directed to measurement of physical parameters, and more particularly, to fast-response circuitry that supports accurate measurement of small sensor changes.

The present invention is applicable to a wide range of medical and nonmedical applications including, but not limited to, control of, or alarms for, physical systems; or monitoring or measuring physical parameters of interest with closed loop feedback. The level of accuracy and repeatability attainable in a highly compact sensing module or device may be applicable to many medical applications that benefits from monitoring or measuring physiological parameters throughout the human body. A closed loop feedback system supports real-time measurement of the parameter of interest and adjustment of the measured parameter by introduction of a change agent that maintains the parameter within a predetermined range. Moreover, the closed loop feedback system can be used in support of or as a supplement to the natural system within a body that normally regulates the parameter. For example, a pancreas is an insulin delivery system in the maintenance of glucose levels. Beta cells of the pancreas delivers microbursts of insulin into the portal venus system which is directly delivered to the liver. The liver is gate keeper of the metabolic system that releases sugar or stores sugar as glucogen. Insulin supports sugar transport. Having too little insulin or too much glucose released can lead to a toxic condition. The present invention eliminates human intervention as part of the feedback loop in determining insulin levels such as measuring glucose levels in blood or responding to glucose measurement via an injection. In either case, the timing of the glucose measurement and the delivery of insulin can vary by control of the patient thereby resulting in non-optimal results.

In one embodiment, a closed loop feed back system comprises glucose measurement and insulin delivery where glucose levels are maintained within a predetermined range. Peripheral insulin delivery, regardless of the means, and regardless of the extent to which peripheral glucose measurements are incorporated, carries with it significant physiological lag. This inefficiency is due to the fact that the insulin must first be absorbed and travel systemically before it arrives at the liver, the hub of metabolic function, rather than the other way around (normal physiology—early autonomic nervous system and sensory perception of an impending glucose challenge, GIT food breakdown, portal transport of glucose and pancreatic secretion of insulin and glucagon to the liver first, then systemic dispersion). As understood from applicable feedback theory analysis, the control loop of the blood glucose measurement and subsequent insulin injection must then require behavioral constraints, constant supervision (external blood testing), and significant outside periodic intervention such as compensation, corrective loop dampening or excitation, calibration, and adjustments for the different constraints that can affect glucose levels. The behavioral constraints, supervision, and intervention comprises the control loop (manual intervention) is the burden of the diabetic patient.

Furthermore, ensuing challenges associated with the patient as the control loop results in excess vascular insulin that is difficult to avoid and leads to fat accumulation, weight gain, and the potential building of insulin resistance, thereby increasing the difficulty in achieving glycemic regulation. Since the sensing of glucose is also peripheral, significant lag in the assessment of relevant sugar levels is experienced (reported to be several minutes or more), thus exacerbating loop control inefficiencies and challenges. Although a patient can be reliable as a control loop there are often times due to normal human activity where the glucose levels can vary substantially. Thus, therapy regulation is sub-optimally reactive, rather than optimally proactive as intended by normal physiology. The patient as a “control loop” moves through periods of being over-damped (too much delivered insulin resulting in trending hypoglycemia), under-damped (too little delivered insulin resulting in trending hyperglycemia), and critically damped (multiple interventions and corrective actions required to achieve an oscillating behavior between the boundaries).

Normal physiology where the pancreas is part of the control loop can be said to be pro-active, driving tight closed-loop control by interjecting many small step inputs into just the right place in the control loop such as the portal venous system. An initial first phase insulin release by the pancreas begins even before food is consumed in response to smell and autonomic nervous system behavior as part of closed loop feedback to anticipate changing glucose levels (this is only partially understood). Subsequent phases of insulin release (as part of a closed loop response) continue in response to GIT and portal venous glucose levels, where there are sugar sensitive receptors (again only recently identified and partially understood) that signal the pancreas accordingly. Portal insulin levels are assessed by the liver which modulates the amount of insulin allowed to disperse systemically, where the insulin is used to trigger (gate or catalyze) glucose transport into cells for metabolic use. Insulin is also used by the liver to convert portal glucose into glycogen, in which it is stored in this form.

Glucagon is released by the pancreas to be used by the liver to breakdown glycogen and release needed glucose for systemic cellular activity as the body demands. These are integrated but separate processes, the timing of which is crucial to optimal control and regulation. Glucagon production and secretion is an on-going process in pancreatic alpha cells that is actually modulated by the level of insulin (normally produced and secreted by neighboring pancreatic beta cells.) Higher levels of insulin suppress glucagon secretion, while lower levels of insulin stimulate an increase in glucagon secretion. This is logical since an increased insulin secretion is normally tied tightly to an increase in portal glucose levels (i.e., at meal-times) when the body does not need or want to break down glycogen. Conversely, low levels of portal insulin are normally tightly tied to low portal (and systemic) glucose levels, and thus needed glucose must be supplied by the breakdown of glycogen, a process for which glucagon is a needed catalyst. In general, the behavior that glucagon secretion is not regulated according to a direct input from a sugar sensitive physiological element, but is rather secondarily moderated by portal insulin concentration. Thus, from a loop control perspective, failure to regulate insulin appropriately has a multiplicative detrimental effect on loop regulation due to its collateral corresponding effect on glucagon secretion. If too much insulin is administered, not only is there a corresponding drop in systemic blood glucose levels (excess blood glucose clearance into tissues, i.e., lipid cell structures), but there is a reduction in glucagon secretion as well, making the resulting hypoglycemia even worse. Conversely, if not enough insulin is administered, glucagon secretion actually increases making the ensuing hyperglycemia even worse (where excess circulating blood glucose causes destructive and toxic reactions). Add to this the fact that a variety of variables such as activity level, patient health and physical condition make normalizing measured glucose values to an appropriate insulin delivery response very difficult without precise and responsive portal insulin concentration management. Determining a peripheral insulin infusion response on the basis of peripheral glucose measurements is therefor grossly sub-optimal, and thus reliable closed loop therapy using these dynamics is never likely, even with significant patient interaction.

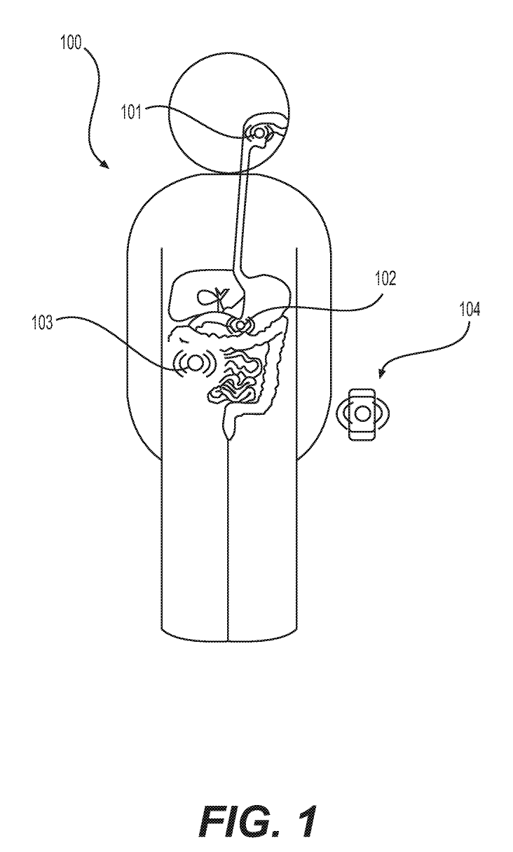

FIG. 1 is an illustration of an artificial pancreas system 100. In general, system 100 is a closed loop system that is placed within a patient's body to monitor a parameter using one or more sensors. System 100 can adjust the parameter in real-time based on measurement data. Thus, system 100 is a closed loop system having one or more sensors that generate quantitative measurement data and adjusting the parameter through some means when outside a predetermined range. System 100 takes into account the physiology that occurs when system 100 delivers a stimulus to adjust the parameter. In the example, an objective of the closed loop, artificial pancreas system is the timely delivery of insulin to the venous system without prior systemic uptake so as to facilitate near normal insulin and glucose processing by the liver, near normal pancreatic alpha cell glucagon secretion management, and the elimination of unwanted systemic control issues and side-effects.

As previously mentioned, system 100 uses one or more sensors to measure parameters of the body. System 100 can comprise one or more devices where each device can have one or more sensors. In the example, system 100 comprises a device 101, a device 102, and a device 104. A device 101 is configured to be placed partially in a mouth or completely in the mouth. Device 101 is configured to measure glucose in saliva of the mouth. Device 101 comprises electronic circuitry coupled to a glucose sensor. The electronic circuitry can transmit measurement data to device 104 or provide measurement data to an outside network for further distribution. As shown, device 104 is outside of the body. In one embodiment, device 104 can be a computer, a mobile device, a tool, or equipment that receives the measurement data. In general, device 101 continuously measures glucose levels while in the mouth. In one embodiment, device 101 is placed completely in the mouth near a saliva duct such that the saliva being measuring is continuously refreshed. Device 101 is designed to stay in the mouth greater than 1 hour to provide continuous glucose measurement data. In other words, it is not a device to take a saliva sample and be removed from the mouth but is intended to stay in the mouth for a period of time and report when the glucose levels in the saliva change. In one embodiment, the measured changes in glucose levels over time are transmitted to device 104. Device 101 has a small form factor and is configured to be comfortable when placed in the mouth and does not interfere with normal mouth activities such as talking, eating, or breathing. Device 101 can be taken out of the mouth for a short period of time when needed and placed back in the mouth to continue glucose measurements. In one embodiment, device 101 is designed to be disposable. A power source within device 101 powers the electronic circuitry for continuous glucose measurements for a predetermined period of time. For example, a power source such as a battery can power device 101 for days or perhaps a week or more. Device 101 can then be disposed of and a new device placed in the mouth for continued glucose measurement prior to the battery being discharged.

In one embodiment, device 104 is in closed loop feedback with device 101. For example, device 104 can be an insulin delivery system. The measurement data from device 101 can support an external insulin pump that is carried by a patient to deliver insulin through a catheter coupled to a vein of the patient. The amount of insulin delivered is based on the glucose measurement data from device 101. Measurement of glucose levels in saliva have been shown to correlate well with glucose levels within the body. The continuous real-time measurements of device 101 can support insulin bursts or micro doses of insulin being provided by the external insulin pump to counter changes in the glucose levels similar to how the pancreas delivers insulin in real-time. Thus, glucose levels can be controlled within a tighter range with continuous sensing and feedback versus the methodology currently used requiring testing a blood sample and injecting insulin in response to the glucose level in the blood sample. The blood sample and injection of insulin is under patient control whereas the continuous detection of glucose in saliva is not. Moreover, an injection can be too large or too small depending on the specific patient situation or status (e.g. just eaten, exercising, etc. . . . ) and when the sample is analyzed since it is a non-periodic or non-continuous measurement situation. Device 101 can include other sensors that can determine patient status along with measurement of glucose levels for better accuracy. In general, closed loop feedback using continuous glucose measurement and mimicking natural insulin delivery as disclosed herein above without requiring patient control can better maintain patient health under varying conditions.

In one embodiment, device 101 can include an insulin delivery system. Device 101 will continuously monitor glucose levels and deliver insulin to change or modify the glucose levels within a predetermined range. Device 101 can be partially in the mouth or completely in the mouth. In the example, device 101 is placed completely in the mouth. In one embodiment, device 101 is placed between the cheek and gum. Alternatively, device 101 can be placed under the tongue. In either example, the insulin delivery system is adjacent to the venous system of the mouth and is located near one or more saliva ducts. In one embodiment, the insulin delivery system sends the insulin system under pressure through tissue in the mouth. A percentage of the insulin will be absorbed by the high density of capillaries found in the tissue where it is efficiently delivered through the venous system similar to how the pancreas delivers insulin. Thus, a closed loop system can be placed in the mouth that continuously measures glucose levels and responds to the measurement data with insulin bursts or micro doses similar to how the pancreas delivers insulin. Thus, the process of measuring glucose levels and delivering insulin is automatic with device 101 and does not involve patient control. Device 101 can include a reservoir for storing insulin. A pump coupled to the reservoir pumps the insulin under pressure into tissue of the mouth. As mentioned previously, the power source can be a limiting factor on how long device 101 can be kept in the mouth to control blood sugar levels. Alternatively, removal of the device can be a function on the reservoir size. For example, the insulin supply in device 101 will last greater than an hour but typically is designed for at least three days or a week before having to remove device 101 for disposal or to refill insulin and replace the power source.

Device 101 can be used with or without device 102. Similar to device 101, device 102 can be used to monitor glucose levels and transmit measurement data to device 104. In one embodiment, device 102 is a glucose monitoring and insulin delivery system configured to be swallowed, move through the GIT, and attach at a predetermined location within the GIT. Device 102 measures glucose in proximity to the portal venous system. Access to the portal venous system is achieved through a GIT (gastro-intestinal tract). In one embodiment, device 102 comprises an ingested capsule having the closed loop system for monitoring glucose levels and adjusting glucose levels by delivering insulin. Artificial pancreas system 100 further includes device 104 coupled to device 102 configured to receive measurement data. The device can include a processor for analyzing the quantitative measurement data and to provide feedback to adjust insulin delivery to the patient. In the example, device 102 includes an insulin delivery system that is a closed loop with continuous glucose measurement that delivers insulin in a manner very similar as the pancreas. Alternatively, artificial pancreas system 100 can be more than one device (device 101 and device 102) located in different areas on a patient body or in different areas within the patient body for monitoring different areas of the body and providing quantitative measurement data from the locations. For example, device 101 is placed in the mouth to monitor glucose levels in saliva and device 102 can be delivered within the GIT to monitor glucose levels in the GIT. Delivery of insulin can comprise both device 101 and device 102 to maintain glucose levels within a predetermined range. Devices 101 and 102 can comprise closed loop systems independent from one another. Measurement data can be provided to device 104 to analyze the effect of devices 101 and 102 and make adjustments since each device will have different effectiveness and lags in delivering insulin to the body. Also, device 104 can use measured parameters on the patient body status to implement algorithmic release from devices 101 and 102 dependent on body measured parameters such as glucose levels, heart rate, temperature, etcetera.

In one embodiment, device 102 comprises an ingestible capsule that houses insulin along with appropriate buffering, absorption enhancing agents, and precise delivery control mechanisms. Electronic circuitry coupled to a glucose sensor and other sensors are housed within the ingestible capsule. The glucose sensor in device 101 is configured to measure GIT glucose concentrations whereas the glucose sensor in device 101 is configured to measure glucose concentrations in the saliva or breath. Electronics inclusive of signal processing and wireless communications in both the oral sensor construct (device 101) and in the ingestible capsule (device 102) facilitate communication between the system constituents for closed loop autonomous system behavior as well as communications to outside data collection, monitoring, calibration and adjustment means (such as device 104). The external communications facilitates integration into a much larger database system for the collection of data, patient experiences and interactions in order to grow most effectively our collective body of knowledge and understanding of how human factors and the artificial pancreas system can control glucose levels and deliver insulin in a response thereto.

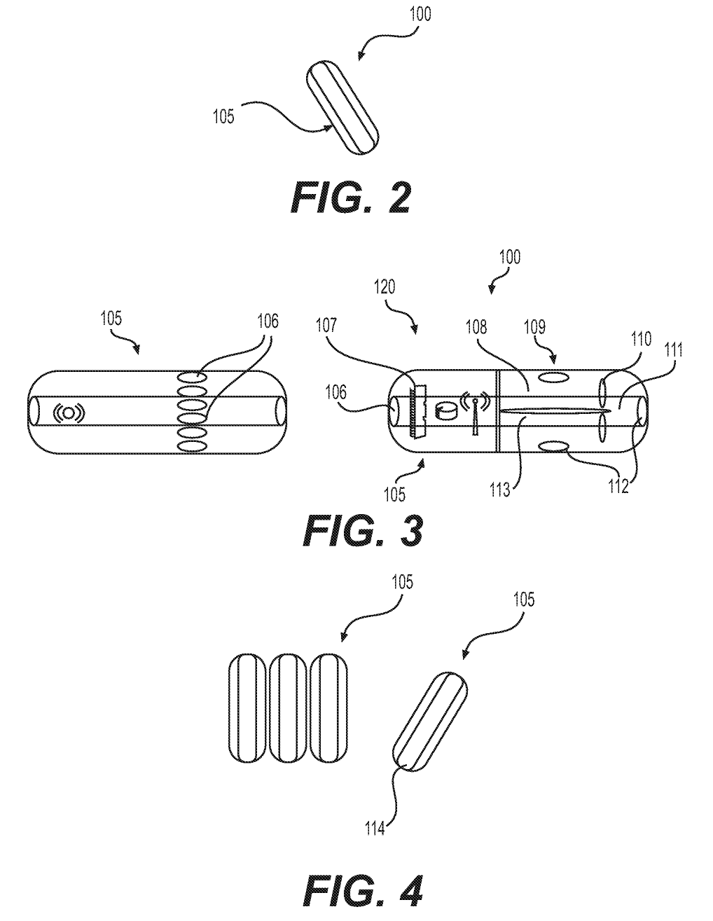

FIG. 2 is an illustration of device 102 in accordance with an example embodiment. System 100 is a closed loop system that is partially or totally placed within a patient's body to monitor one or more parameters using one or more sensors and adjusting the one or more parameters based on the quantitative measurement data. An ingestible capsule 105 includes electronic circuitry and one or more sensors. In the example embodiment, capsule 105 comprises insulin storage and means for precise micro-volume delivery. The electronic circuitry can include communication circuitry to transmit and receive information. The electronic circuitry controls a measurement process and controls delivery of the insulin. Typically, the size of ingestible capsule 105 is equal to or less than 18 millimeters in length, 12 millimeters, 12 millimeters in width, and 5 millimeters in height to support swallowing of the device.

FIG. 3 is an illustration of ingestible capsule 105 illustrating electronic circuitry 120 to measure a parameter and form a closed loop that adjusts the parameter based on quantitative measurement data in accordance with an example embodiment. Ingestible capsule 105 comprises a glucose measurement system, additional sensors, and an insulin delivery system. Moreover, ingestible capsule 105 is designed to mimic a pancreas for precise micro-volume delivery of insulin. In one embodiment, micro-volume delivery of insulin is achieved by the dissolution of layered or nested enteric coatings. Electronic circuitry 120 comprises digital logic 107 for controlling a measurement process. Digital logic 107 can comprise digital logic, an ASIC (application specific integrated circuit, a processor, a DSP (digital signal processor), standard cells, and logic gates/logic structures. Electronic circuitry 120 can further include analog circuitry, interface circuitry, conversion circuitry, memory, power management circuitry, or communication circuitry to support measurement of glucose levels and other parameters. In general, a reservoir of insulin is stored in or on ingestible capsule 105 that can be released under control of electronic circuitry 120. In the example, insulin is stored on a surface of ingestible capsule 105 or between layers formed on the device.

A reservoir 111 stores insulin within ingestible capsule 105. The reservoir can store insulin to maintain glucose levels within a predetermined range for days or weeks. In one embodiment, electronic circuitry 120 couples to electrodes 106, electrodes 112, and a transducer 113 for providing ultrasonic stimulus. Electrodes 106 and 112 are configured to provide a voltage, a current, or variable time/magnitude voltage or current. In one embodiment, the voltage, current, or ultrasonics are coupled to the one or more layers that retain and store the insulin. Dissolution of the one or more layers controls results in the release of insulin.

In one embodiment, three or more structures or layers are used to control the release of insulin. At least one layer comprises an enteric coating to protect the insulin from disintegration in a gastric environment. As shown, structures or layers 108, 109, and 110 prevent insulin from being released within the GIT. Structures or layers 108, 109, and 110 can be controlled (catalyzed, enhanced or suppressed) by electrodes 106 or 112 and/or transducer 113 from inside the ingested device. The insulin (inclusive of possible buffering, transport and absorption enhancement agents) is trapped, stored and protected in between nested layers. Controlled and successive dissolution of the nested coatings from the outside-in releases micro-volumes of insulin layer by layer. Dynamic, time-variant regulation of the entire process is under algorithmic control that uses saliva, systemic, and GIT glucose sensing, pH sensing, temperature sensing, and other sensors for measuring parameters of within the body. For example, one coating or layer can have pore structures that relax and open more with ultrasonic stimulus. Another layer type can fracture and break apart with increasing osmotic pressure. The fracturing can be facilitated by the layer being catalyzed by electric field modulation of the fluid outside the capsule which results in water being drawn into the layer. In one embodiment, the outermost coating may be intended to allow the capsule to move from the stomach into the GIT before glycemic regulating function begins. Indeed, by varying the thickness of the outer coating, the location in the GIT where function begins can be targeted, adjusted or simply made variable.

One of the structures or layers 108, 109, or 110 can have properties that encourage “adhesion” to the mucous rich tissues inside the GIT, whereby the variable dissolution of an outer enteric coating facilitates targeted or variable temporary adhesion to the inside of the GIT for the purpose of slowing down progression through the digestive system and bringing the capsule into more intimate contact with the epithelial structures, thereby enhancing insulin transport, protection and uptake. A network of electrodes 106 and 112 placed around ingestible capsule 105 supports low level electric field modulation for the purpose of opening the absorption channels and facilitating more effective insulin transport into gastric capillaries that feed directly into the portal venous system. Structure or layer 108, 109, or 110 of ingestible capsule 105 can apply local ultrasound stimulation. In one embodiment, ultrasound stimulation using transducer 113 will increase local cavitation and associated disruption of the lipid structures to promote increased efficiency in delivering large molecule transport such as insulin. The electrodes 112 on in and around ingestible capsule 105 can also be used for the purpose of aiding in the buffered protection of the insulin molecules in the GIT by locally modulating pH. A pH sensor can be included in the ingestible system so as to monitor the effectivity of this behavior and track the capsule's progression through the GIT. Ingestible capsule 105 can further include electric field and ultrasonic stimuli that supports the flow of released insulin to the epithelial structures. The combination of these nested coatings offers the ability to dynamically control micro-delivery of insulin without the need for more challenging and expensive technologies.

FIG. 4 is an illustration of ingestible capsule 105 illustrating coatings that store and release a chemical in accordance with an example embodiment. Ingestible capsule 105 comprises a glucose measurement system, additional sensors, and an insulin delivery system. Moreover, ingestible capsule 105 is designed to mimic a pancreas for precise micro-volume delivery of insulin. In one embodiment, micro-volume delivery of insulin is achieved by the dissolution of layered or nested enteric coatings. Electronic circuitry 107 of FIG. 3 can control the dissolution of the one or more layers. In one embodiment, layers 114 dissolve from the outside in. An outermost layer can support the passage of ingestible capsule 105 through the stomach and GIT without breakdown of the device or delivery of insulin. A layer underlying the outermost layer can support attachment to tissue within the small intestine. Ingestible capsule 105 can further include one or means such as chemicals, coatings, or electrical stimulation to adhere to tissue within the small intestine in proximity to the portal venous system. Ingestible capsule 105 measures glucose levels within the small intestine and delivers insulin to the portal venous system similar to how the pancreas delivers insulin.

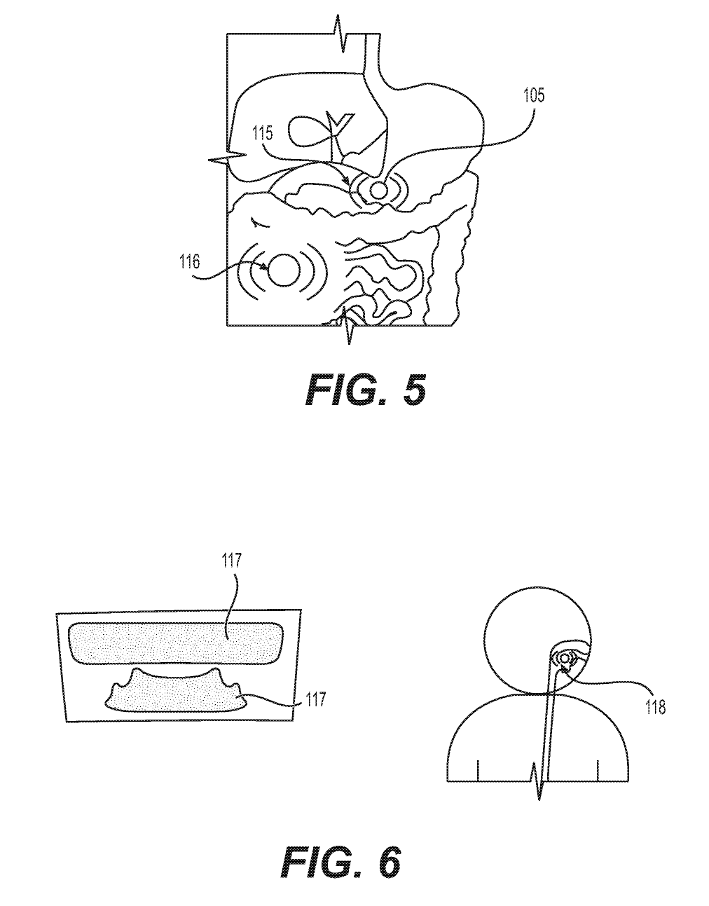

FIG. 5 is an illustration of ingestible capsule 105 passing through the GIT where one or more sensors activate when approaching a predetermined location in accordance with an example embodiment. In the example, ingestible capsule 105 comprises a GIT glucose sensor and an insulin delivery system coupled in a closed loop to maintain glucose levels within a predetermined range. Ingestible capsule 105 is configured to activate the one or more sensors therein after leaving the stomach and passing the duodenum 115. Ingestible capsule 105 continues to move through the GIT from the duodenum 115 to the small intestine 116. In the small intestine 116, ingestible capsule 105 will begin to release insulin based on the quantitative measurement data generated by the GIT glucose sensor. In general, the closed loop artificial pancreas system disclosed herein above relates to the sensing of a glucose challenge facilitating a tight closed loop response to the measured glucose level. Recognizing that glucose uptake in the GIT involves markers that can be observed, ingestible capsule 105 includes a glucose sensor configured to measure GIT glucose in possible combination with other markers that allow assessment of the magnitude of the glucose challenge and its time-variant behavior, influenced by GIT uptake dynamics. The closed loop system can include software and algorithms based on quantitative measurement data that addresses concerns over GIT process variability influenced by one or more factors, including the magnitude of the load and the health of the patient. The artificial pancreas system can account for the time variant behavior of GIT glucose levels and other process markers that facilitates real time assessment of glucose uptake and entry into the portal venous system, thereby emulating normal physiology and making closed loop control possible. A GIT sensor interface can be protected by an enteric coating that dissolves once the capsule has passed from the stomach into the duodenum 115 and progresses through the small intestine 116. A semi-permeable polymer can underlie the enteric coating that is exposed when the enteric coating dissolves. The semi-permeable membrane can be resistant to GIT fluids but permeable to glucose to further protect the sensor interface as it begins to perform. A sensor construct can comprise a configuration of glucose-oxidase, platinum or gold electrode layers. Since sensing glucose challenges in the GIT has primarily a predictive benefit, discerning changes and their time-varying relative magnitudes is of primary importance thereby facilitating a pragmatic implementation of an adaptive algorithmic approach.

FIG. 6 is an illustration of a sensor interface comprising one or more sensors in accordance with an example embodiment. A sensor array 117 comprises a glucose sensor, a temperature sensor, and a breath analyzer. Sensor array 117 can also include other sensors. Referring briefly to FIG. 1, sensor array 117 can be part of device 101 or device 102. Sensor array 117 is controlled by electronic circuitry within device 101 or 102 and can transmit quantitative measurement data to device 104. An oral saliva sensor of sensor array 117 offers a very early glucose challenge indicator that can be used algorithmically to initiate a very early first stage insulin delivery response. While real time assessment of the magnitude of the glucose challenge is critical for a tight closed loop response, just as important is a real time assessment of systemic glucose clearance from the blood into the tissues by measuring the circulating blood glucose concentration, or a strongly correlative analog. Research has shown unchallenged saliva glucose concentrations to be strongly correlative to that of blood glucose concentrations. In general, saliva glucose concentrations without the presence of food in the mouth will settle to levels that are correlative to circulating blood glucose concentrations whereby a saliva glucose sensor in the mouth offers system level real-time glucose assessments. Any physiological lag that may exist between circulating blood glucose concentrations and those measured in saliva is more than compensated for by the predictive early assessment value that a saliva sensor brings to a glucose challenge. In one embodiment, the saliva sensor is housed in a very thin and narrow construct such as sensor array 117 that can be applied and secured between the teeth and cheek. A capture mechanism that fits over a molar is also possible as another embodiment option. Similarly, a thin under the tongue device might also be useful for saliva access. All can be placed in the mouth comfortably for greater than one hour and realistically for days or weeks. A mouth guard embodiment might be useful especially for glucose monitoring during sleeping hours. In the example embodiment, a saliva sensor 118 is placed between the cheek and gum as disclosed herein in above which can support normal mouth activities such as each eating, talking, or breathing since it is out of the way or does not impact these activities. In one embodiment, saliva sensor 118 can include a temperature sensor so as to monitor body temperature and adjust loop dynamics accordingly. Note that body temperature can be an indicator of health challenges, exercise and metabolic activity. Additionally, it is possible to include in the same construct as that used to house saliva sensor 118 an additional sensor means for detecting high or low systemic glucose concentrations by breath analysis. This sensor means would serve as another confirming systemic glucose measurement, particularly useful in catching abnormal highs and lows. In this embodiment, breath analysis is miniaturized and targeted for alarming when a potentially dangerous high or low systemic glucose condition exists. This additional indicator offers a valuable back-up alert, and a pre-programmed closed loop corrective response. With further development and experience, this sensor means could be integrated into normal loop dynamic assessment and regulation algorithms.

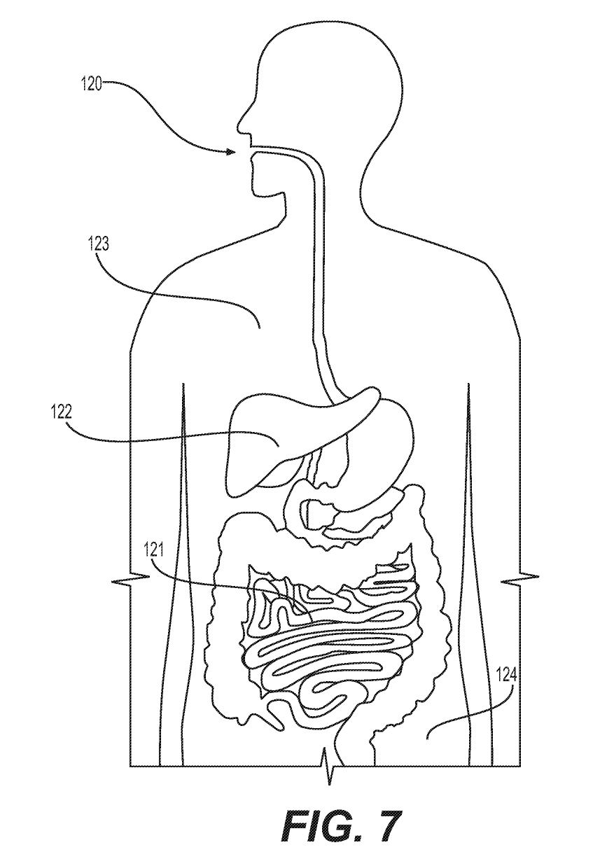

FIG. 7 is an illustration of the mouth and GIT in accordance with an example embodiment. A primary objective of any closed loop artificial pancreas system must be an effective control algorithm that not only facilitates as closely as possible system dynamics that emulate normal physiology, but also integrates into the analysis real-time assessment of key input variables (such as the level of exercise/activity, temperature) and allows for learning adjustments autonomously and/or via outside wireless intervention. The interaction between the various physiological processes is first disclosed through a compartmental analysis that describes the changes in glucose and hormonal processes at natural physiological boundaries within a body. In one embodiment, the system can be described as a set of differential equations as shown herein below, descriptive of boundary changes in response to a glucose challenge that ultimately can be observed as a change in circulating blood glucose concentrations.

IDR˜C0*Gm+C1*(d1Gm/dt)+C2*(d2Gm/dt)+C3*(d3Gm/dt) where IDR is an insulin delivery rate, C0-C3 are coefficients, Gm is measured glucose concentration and d/dt is a derivative with respect to time.

Consider a first compartment 120 that comprises an oral cavity or mouth 125 which breaks down food intake and GIT 121. First compartment 120 has a natural first boundary between the GIT and the portal venous system. First compartment 120 includes processes that releases nutrients which are then taken up and transported to the liver. A second compartment 122 comprises both the liver and the pancreas. Second compartment 122 includes processes that are governed by the changes observed at the first boundary along with changing sensory inputs such as sugar sensors in first compartment 120 and autonomic nervous system inputs. Consider a third compartment 123 to be the systemic circulatory system where circulating blood glucose levels are a function of the changes at the second compartment boundary, driven by liver and pancreatic processes, as well as the changes at the boundary with a fourth compartment 124. Fourth compartment 124 comprises cells that need glucose for metabolic function. Thus, there are four compartments 120, 122, 123, and 124 with three boundaries between them where there are dynamic differential changes in glucose concentration, leading to a descriptive model comprising equation 125 that includes the current blood glucose concentration in summation with the first, second and third order differential changes in blood glucose concentrations to more fully capture the dynamics behind what can be observed in the blood. In this way a more predictive and responsive insulin delivery algorithm can be formulated that has the ability to emulate appropriately normal physiology, especially if and when insulin delivered in this way enters the portal venous system directly as our bodies intend. The weighted coefficients of each of the terms such as C0-C3, measured glucose concentration+first+second+third order derivatives of the measured glucose concentration with respect to time, can be dynamically adjusted to optimize loop regulation. Indeed, not only can the weighting be adjusted as a part of an initial calibration process, but it can be dynamically adjusted through a learning algorithm that interprets hypo or hyper-glycemic trends and, taking the magnitude of the deviations from target into consideration, make small iterative adjustments to minimize the error term. Also of note is the possibility to tailor the algorithm to adjust for other variables such as activity, temperature, sleep and fasting. By understanding where in the model these variables have an impact, the coefficients to the appropriate terms can be adjusted. For example, when preparing for sleep and a several hour fast, the coefficient for the third order differential can be de-weighted since food breakdown in the GIT will not be in play, and the algorithm must therefore prioritize those terms that describe second and third boundary changes, recognizing that inputs into the process take place in the second compartment (pancreas and liver) only, and not the first. The adjustments can be made autonomously by tracking systemic glucose trends, by pre-programmed time of day synchronization, by patient interaction with a small wireless programming device or phone application, or even by a distinct capsule type for ingestion prior to bedtime. As well, in the case of exercise and activity, inputs from accelerometers, gyroscopes, and/or piezoelectric transducers already available in external devices (i.e., mobile phones) along with simple patient initiated programming interventions, can be used to assess activity level and adjust coefficients accordingly through wireless programming adjustments. In this case, it may be optimal to add weight to the first order and proportional terms since glucose clearance may carry more influence due to metabolic demand. It can therefore be seen how through experience one can learn how to best dynamically adjust the algorithm and achieve near normal loop regulation dynamics. Of particular interest is the strong benefit that comes with both sensing glucose concentration in the saliva and fluid in the GIT. Saliva glucose measurements not only offer an understanding of system glucose concentrations and associated glucose clearance after food has been ingested, but as well, saliva concentrations offer the opportunity for very early first phase insulin response, helping to mimic normal physiology inclusive of the predictive behaviors as closely as possible. This approach can strongly emulate normal physiology by summing the predictive elements with the present state. The summation coefficients can be autonomously or interactively adjusted to achieve the best closed loop dynamics on a patient by patient, system by system, and even real time basis. The integration and weighting of independent sensor sources, oral and GIT, along with a weighted assessment of exercise and activity level (via accelerometer and/or gyroscope technology, or piezo-electric activity monitoring) and other environmental, physiological influences (i.e., temperature) offers the potential for sophisticated and robust control.

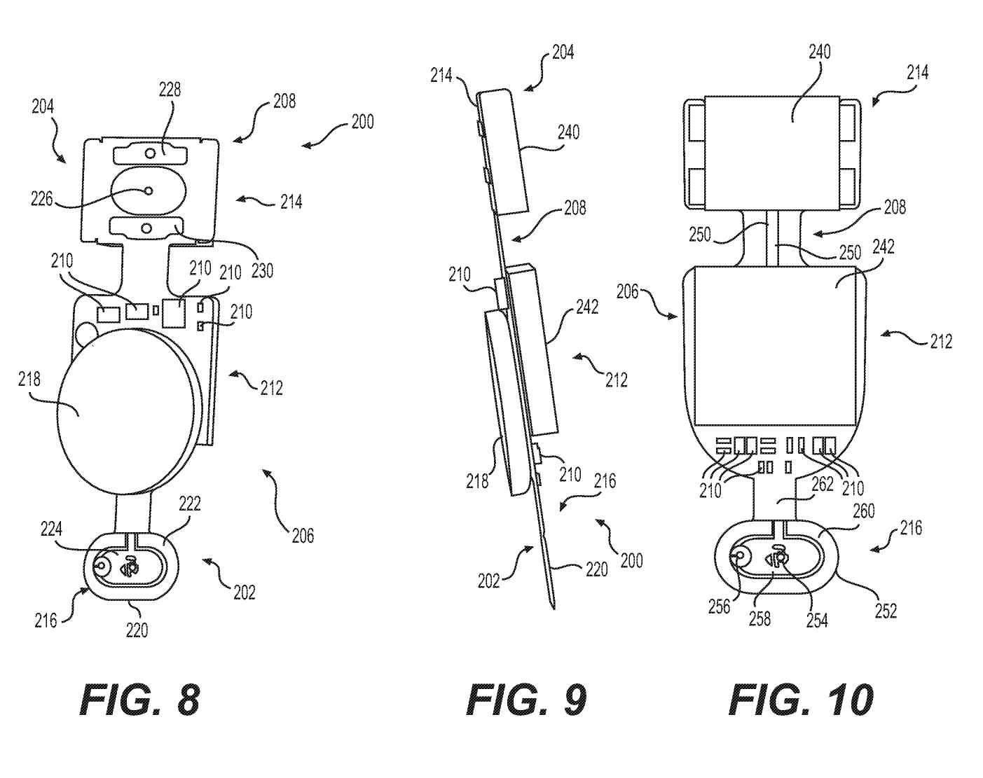

FIG. 8 is an illustration of an artificial pancreas system 200 in accordance with an example embodiment. Artificial pancreas system 200 corresponds to artificial pancreas system 100 and can include electronic circuitry, coatings, and sensors of device 101 and 102 shown in FIG. 1. Artificial pancreas system 200 comprises a glucose measurement system 202 and an insulin delivery system 204. In one embodiment, the glucose measurement system 202 is a continuous measurement system that can be used in a closed loop with an external insulin deliver system. As shown, artificial pancreas system 200 is a closed loop system that measures glucose levels and responds to the glucose measurements by delivering microbursts of insulin, for example in nano-liter quantities over multiple delivery cycles, until a predetermined amount of insulin has been delivered. In general, the liver does not want to see a continuous feed of insulin that corresponds to the common method of injecting insulin.

Glucose measurement system 202 can be used in the mouth to measure glucose in saliva or in the GIT (gastro-intestinal track) to measure glucose in the intestinal track. In one embodiment, an external insulin delivery system would provide insulin based on the glucose measurement data transmitted from glucose measurement system 202. Glucose measurement system 202 measures glucose level continuously or periodically and can transmit the glucose measurement data and other sensor data remotely. In the example, the insulin delivery system 204 is a device placed within a patient body to deliver insulin to a patient venous system. The insulin delivery system 204 can be used in conjunction with an external glucose monitoring system to support insulin delivery. Insulin delivery system 204 can be placed in the mouth to deliver insulin to the venous system in the mouth or to deliver insulin to the portal venous system within the GIT similar to the pancreas. In a third embodiment, artificial pancreas system 200 is a self-contained closed loop system comprising glucose measurement system 202 and insulin delivery system 204. Artificial pancreas system 200 can be configured to be placed in the mouth to monitor glucose in saliva and deliver insulin to the venous system in the mouth. Similarly, artificial pancreas system 200 can be configured to be swallowed by a patient to monitor glucose in the GIT and deliver insulin to the portal venous system in proximity to the GIT. In general, artificial pancreas system 200 is configured to be kept in the mouth for greater than 1 hour and typically several days to perhaps two weeks. Artificial pancreas system 200 is a disposable system that can be removed from the mouth or leaves the GIT after a predetermined time. After disposal, a new artificial pancreas system can then be placed within the mouth or delivered to the GIT to maintain glucose levels within a predetermined range. In a fourth embodiment, a first artificial pancreas system 200 is placed in the mouth and a second artificial pancreas system 200 is placed in the GIT. Glucose is configured to be monitored in both the GIT and within saliva of the mouth. The first and second artificial pancreas systems 200 are used in concert to maintain patient glucose levels. Indicators in saliva and the GIT can be used to determine the state of the body (e.g. resting, exercising, preparing to eat etc. . . . ) and provide varying levels of insulin delivery into the venous system or the portal venous system taking into account the differing lag times and efficiency differences in the insulin delivery to maintain glucose levels within the predetermined range.

In general, artificial pancreas system 200 is configured as a closed loop system that can measure glucose levels in a fluid and deliver insulin in proximity to the glucose measurement. It should be noted that artificial pancreas system 200 can be separated into a separate glucose measurement device and a separate insulin delivery system for use as disclosed herein above. In the example, electronic circuitry 206 is configured to control glucose measurement and insulin delivery in a closed loop and in proximity to one another. Thus, electronic circuitry 206 couples to and controls a glucose sensor and an insulin pump. Separate electronics are required if glucose measurement and insulin delivery are not in proximity to one another. In one embodiment, artificial pancreas system 200 comprises a single device that is placed in the mouth or in the GIT. Electronic circuitry 206 is also configured to transmit glucose measurement data or insulin delivery data to a remote device or a computer. The glucose measurement data or insulin delivery data can be stored by electronic circuitry 206 in local memory or on non-local memory such as a data server. In one embodiment, glucose measurement data or insulin delivery data is encrypted before transmission to prevent access should the transmission be intercepted.

In the example, artificial pancreas system 200 comprises a flexible interconnect printed circuit board 208. Printed circuit board 208 can have more than one layer of interconnect. A first side of printed circuit board 208 includes interconnect for coupling components 210 and digital logic together to form a circuit for controlling a measurement process and to support delivering medicine. Components 210 can be mounted on the first side and a second side of printed circuit board 208. A majority of electronic circuitry 206 is coupled to a first section 212 of printed circuit board 208. In the example, first section 212 is a central portion of printed circuit board 208. A power source 218 is coupled to the first side of first section 212 of printed circuit board 208 to provide power to electronic circuitry 206. In one embodiment, power source 218 can be a battery. Alternatively, power source 218 can be an inductor, capacitor, or other power source configured to power electronic circuitry 206 for a predetermined time period.

A first side of second section 216 of printed circuit board 208 comprises a glucose sensor 220. Glucose sensor 220 is a non-invasive sensor that does not require a skin puncture to continuously monitor glucose levels. In one embodiment, a second glucose sensor can be on a second side of second section 216 of printed circuit board 208. Having at least two glucose sensors can provide redundant glucose measurements, provide a second sensor to extend a period over which glucose can be measured, or to operate each glucose sensor during a period of optimal sensitivity and measurement accuracy. Electronic circuitry 206 can have a switch to enable each glucose sensor individually or together to provide glucose measurement data. In the example, glucose sensor 220 is configured to be bathed in a fluid such as saliva or fluid in the GIT such that glucose levels can be measured continuously or periodically. In the example, glucose sensor 220 is configured to be placed between a cheek and gum of a patient such that glucose sensor 220 is near a saliva duct. Saliva transport is over the surface of glucose sensor 220 that is continuously replenished as saliva is delivered from the saliva duct and older saliva is removed or swallowed from the mouth. Alternatively, glucose sensor 220 can be placed under the tongue whereby saliva is introduced to a surface of glucose sensor 220, removed, and replenished. Glucose sensor 220 can comprise two or more electrodes and include one or more chemicals configured to support glucose measurement. In one embodiment, glucose sensor 220 comprises a working electrode 224 and a counter electrode 222. The one or more chemicals can be coupled to the surface of glucose sensor 220 and can be configured to dissolve over time as glucose is measured. In one embodiment, the chemistry is coupled to working electrode 224. For example, the one or more chemicals can be printed or layered onto a metalized pad of working electrode 224. In one embodiment, counter electrode 222 does not have any chemicals on a metalized pad of counter electrode 222. In general, working electrode 224 and counter electrode 222 have a predetermined spacing between the metalized pads. In one embodiment, a voltage bias is applied across working electrode 224 and counter electrode 222. In the example, working electrode 224 is coupled to ground and a voltage is applied to counter electrode 222. A current is conducted between counter electrode 222 and working electrode 224 that can include ions from the one or more chemicals introduced to the saliva. Furthermore, the one or more chemicals on working electrode 224 can react with glucose in the saliva. The current flowing from counter electrode 222 to working electrode 224 corresponds to the amount of the glucose in the saliva. In one embodiment, the glucose is continuously measured or measured with predetermined time intervals such that changes in glucose levels can be reacted to by artificial pancreas system 200 to maintain glucose levels within a predetermined range or to adjust based on patient status or activity. The glucose measurement data can be analyzed by electronic circuitry 206 to adjust insulin delivery in real-time, can be stored in memory on artificial pancreas system 200, or transmitted to a device exterior to the patient.

A three electrode glucose sensor can also be used. The three electrode glucose sensor comprises a working electrode, a counter electrode, and a reference electrode. The working electrode can have one or more layers of chemistry to facilitate glucose measurement in saliva or glucose measurement in fluids of the GIT. A bias voltage can be applied to the counter electrode to determine current flow such that glucose levels can be accurately measured. The reference electrode is configured to reference the working electrode and the counter electrode. The reference electrode can be modulated for measurement accuracy or changing chemistry. Similarly, the reference electrode can be adapted to support recalibration of glucose measurement electrically. In general, continuous glucose monitoring is less invasive and has less side effects that monitoring glucose with a catheter beneath the skin. The size of glucose sensor 220 corresponds to measurement of lower glucose levels but can easily fit between cheek and gum of the mouth.

A first side of third section 214 of printed circuit board 208 comprises a portion of insulin delivery system 204. Insulin delivery system 204 comprises an outlet port 226, an electrode 228 and an electrode 230. Outlet port 226 is configured to deliver insulin or another medicine. In one embodiment, outlet port 226 is placed adjacent to tissue in the mouth or tissue within the GIT. A pump couples to outlet port 226 to deliver the insulin. Insulin comprises a large molecule that will breakdown over time if not delivered to the liver. Ideally, the insulin is delivered to the portal venous system which couples to the liver and is similar to how the pancreas delivers insulin. Alternatively, the insulin can be delivered to the venous system albeit at a slight loss in efficiency before being delivered to the liver. The insulin would be pumped through the heart before being delivered to the system thereby introducing lag. Artificial pancreas system 200 can deliver insulin to the portal venous system when placed in the GIT. In one embodiment, outlet port 226 is placed adjacent to buccal tissue in the mouth. Buccal tissue has a capillary network that is not too distant from the surface of the tissue in the cheek of the mouth. Outlet port 226 couples to a pump for delivering insulin. In one embodiment, insulin is pumped near or on the buccal tissue where a portion of the delivered insulin is absorbed by the capillary network below the surface of the tissue of the cheek such that the insulin is delivered through the venous system to the liver. Buccal tissue has a large capillary network beneath the surface tissue in the mouth. Artificial pancreas system 200 includes pumping methods and electrical stimulation methods to place the insulin much closer to the capillary network within and beneath the buccal tissue. In one embodiment, channels can be formed in the buccal tissue to more efficiently place the insulin closer to the capillary network thereby by increasing the efficiency of insulin absorption and reducing lag. A first method for forming channels or placing the insulin closer to the capillary network is to pump, push, or pulse the insulin at high frequency. For example, a 125 hertz pump cycle can be configured to deliver a predetermined volume of insulin. In one embodiment, the 125 hertz pump cycle comprises a fill cycle and a delivery cycle. In one embodiment, the insulin can be excited ultrasonically. For example, the pump can be can be operated at ultrasonic frequencies during the delivery of insulin or after the insulin is delivered to pulse or vibrate insulin or saliva into the buccal tissue or into channels formed by the pulsed liquid. For example, the insulin can be pulsed at an ultrasonic frequency such as 1-1.5 megahertz. In one embodiment, pulsed or vibrated insulin can disrupt the buccal tissue to form channels in which the insulin can flow. The channels are disruptions or gaps in the buccal tissue that are closer to the capillary network. Note that the pump output is adjacent or near to the buccal tissue. Thus, the channels or gaps are also formed where the insulin is delivered. The insulin is driven into the channels near the network capillaries below the tissue surface to be absorbed by the venous system and the insulin delivered to the liver before breaking down.

A second method to support transport of the insulin to the network of capillaries in the mouth is to apply a voltage across electrodes 228 and 230. In one embodiment, the voltage can be modulated to produce a repeating voltage change across electrodes 228 and 230. The changing voltage across electrodes 228 and 230 generate a varying electric field that disrupts fat cells in the epithelial boundary to open up channels in the fat system to increase absorption of large molecules. Similar to the ultrasonic excitation of the insulin or saliva, the electrical stimulation is micro targeted to the sight where insulin is being released. Note that the electrodes 228 and 230 are placed on opposing sides of outlet port 226. Similar to the ultrasonic disruption disclosed herein above, the electrical stimulation can separate tissue or support a channel forming process to expose the capillary system in the mouth. In one embodiment, both high frequency excitation of insulin or saliva in conjunction with electrical stimulation is used to form channels and enhance large molecule exposure to the dense capillary system in the mouth or GIT to results in rapid absorption into venous system.

FIG. 9 is an illustration of a side view of a portion of artificial pancreas system 200 in accordance with an example embodiment. In general, the side view illustrates the form factor that supports placement of artificial pancreas system 200 between the cheek and the gum in the mouth for glucose monitoring in a closed loop with insulin delivery. The form factor also supports placement within a pill that can be swallowed to measure glucose in the GIT and deliver insulin to the portal venous system. A pump 240 is mounted on the second side of third section 214 of printed circuit board 208. A device such as microprocessor, micro-controller, ASIC, digital signal processor, or a digital logic circuit is coupled to the second side of first section 212 of printed circuit board 208. In one embodiment, the device is a micro-controller 242 configured to control glucose measurement in a closed loop with insulin delivery in the mouth or GIT. Micro-controller 242 couples to glucose measurement system 202, insulin delivery system 204 and electronic components 210. In one embodiment, microcontroller 242 includes communication circuitry to transmit measurement data or receive information. A maximum thickness of artificial pancreas system 202 occurs in a region of the first section 212 of printed circuit board 208 where power source 218 and micro-controller 242 are mounted. In one embodiment, the maximum thickness is less than 6.5 millimeters thick. A minimum thickness occurs where printed circuit board 208 does not have a mounted components or in second section 216 of printed circuit board 208 where glucose sensor 220 resides. As mentioned previously, artificial pancreas system 200 is configured to be placed in the mouth or GIT for more than one hour where changes in glucose levels can be continuously or periodically monitored and insulin provided in a closed loop based off of the glucose measurements or other sensor measurement data. Typically, artificial pancreas system 200 can measure glucose and deliver insulin for days or weeks non-invasively.

FIG. 10 is an illustration of the second side of printed circuit board 208 in accordance with an example embodiment. Micro-controller 242 is shown mounted on the second side of first section 212 of printed circuit board 208. Electrical components 210 are also mounted on the second side of first section 212 of printed circuit board 208. In one embodiment, an antenna can be formed as part of the interconnect of printed circuit board 208. Pump 240 is mounted on the second side of third section 214 of printed circuit board 208. In one embodiment, pump 240 is a piezo electric diaphragm pump configured to deliver insulin in small or tiny bursts. The voltage magnitude and the duty cycle of the input voltage to pump 240 determines the amount of insulin delivered. Printed circuit board 208 has interconnect 250 coupling electronic circuitry 206 to pump 240 to control pump operation.

As mentioned previously, a glucose sensor 252 can be formed on a second side of second section 216 of printed circuit board 208. Thus, artificial pancreas system 200 has a first glucose sensor 220 and a second glucose sensor 252 that can be used redundantly, to maintain optimal measurement sensitivity, or to extend a time frame for continuous glucose measurement. Glucose sensor 252 comprises a working electrode 258 and a counter electrode 260. Glucose sensor 252 is bathed in saliva or GIT fluid to measure glucose. Alternatively, glucose sensor 252 can include a “wicking” material such as cotton gauze to capture saliva for measurement. In the example, glucose sensor 252 is placed near a saliva duct in the cheek or under the tongue of the mouth. Similar to glucose sensor 220, the working electrode 258 is configured to be coupled to ground and can include one or more chemical layers thereon to support glucose measurement. Counter electrode 260 is configured to receive a voltage. A measured current from counter electrode 260 to working electrode 258 corresponds to a glucose level in saliva or fluids of the GIT. In one embodiment, working electrode 258 can be coupled to working electrode 224 on the first side of the second section 216 of printed circuit board 208 by a thru hole via 254. In one embodiment, counter electrode 260 can be coupled to counter electrode 222 on the first side of the second section 216 of printed circuit board 208 by a thru hole via 256. Alternatively, counter electrode 260 and working electrode 258 can be controlled independently from counter electrode 222 and working electrode 224. An interconnect 262 is configured to couple counter electrode 222 to electronic circuitry 206 on the second side of printed circuit board 208.

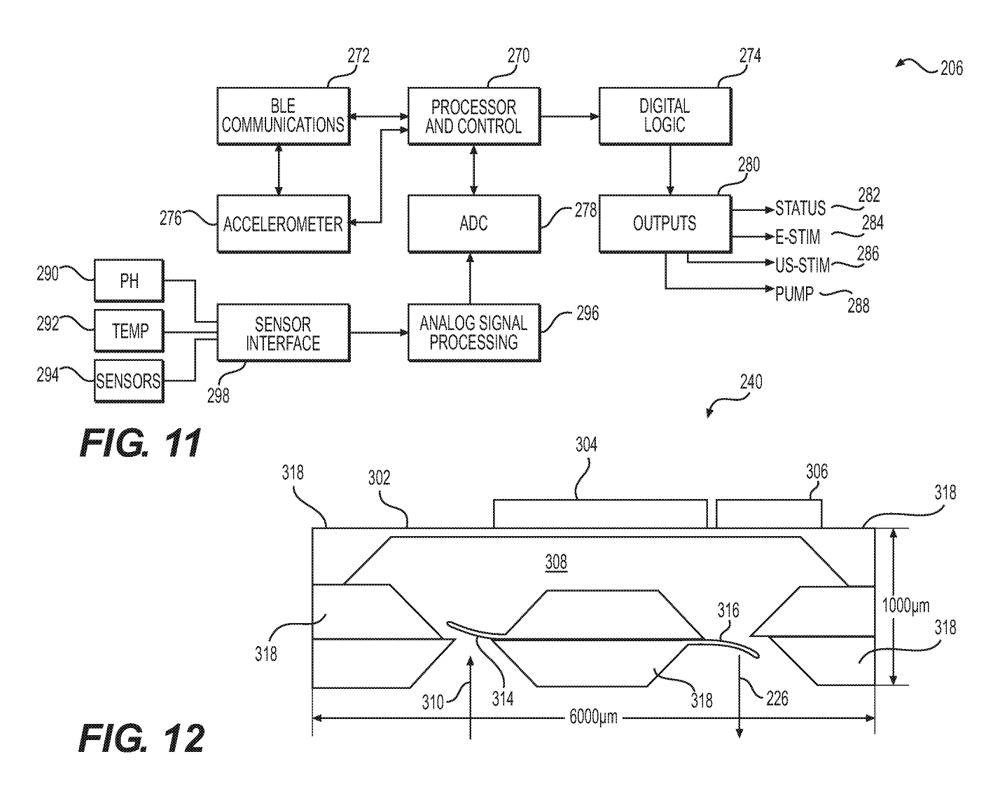

FIG. 11 is a block diagram of electronic circuitry 206 in accordance with an exemplary embodiment. Electronic circuitry 206 of FIGS. 8-10 comprises a processor 270, communication circuitry 272, digital logic 274, accelerometer 276, outputs 280, ADC (analog to digital converter) 278, analog signal processing 296, sensor interface 298, pH sensor, 290, and temperature sensor 292. Processor 270 is configured to control a measurement process and respond to measurement data in a closed loop by providing one or more medicines to produce change in the measurement data. Processor 270 can be a microprocessor, a micro-controller, a digital signal process (DSP), a processor, an ASIC (application specific integrated circuit), or digital logic. Communications circuitry 272 couples to processor 270. Communications circuitry 272 is configured to provide two-way communication with an external device such as a computer, a smart device, or other devices such as a medical device. In one embodiment, communications circuitry 272 is configured maintain a closed loop with an external device. In one embodiment, communications circuitry 272 is a Bluetooth bi-directional communication circuit that is low power to extend an operating life of the artificial pancreas system. The Bluetooth communication can transmit and receive information with a device external to a patient body when the artificial pancreas system is in the mouth or the GIT. In one embodiment, communications circuitry 272 is integrated with processor 270. Communications circuitry 272 can be a wired or wireless connection and is not limited to Bluetooth and can include any other wireless technology or platform. Communications circuitry 272 can further couple to the internet to communicate to one or more devices.

Processor 270 includes one or more inputs from sensors that generate measurement data. An accelerometer 276 couples to processor 270 to measure motion of the artificial pancreas system. In one embodiment, accelerometer 276 is configured as an activity monitor to measure patient status such as exercising or resting. Accelerometer 276 can further be used to detect a vibration such as chewing in the mouth to measure the onset of a glucose load from eating. In one embodiment, accelerometer 276 has digital outputs that can directly be coupled to processor 270.

A pH sensor 290, a temperature sensor 292, or one or more sensors 294 can be coupled to a sensor interface 298. Temperature sensor 292 or pH sensor 290 can be used to monitor patient status. The patient status can relate to glycemic regulation such as if the patient is sick, running a fever, or impacted by environmental factors. Patient status can further include patient activity as disclosed herein above. Sensors 294 can be any other sensor used in the artificial pancreas system. For example, sensors 294 can include an enzyme detector sensor, an insulin concentration sensor, breath analyzer, fructose sensor, sucrose sensor, or cortisol sensor to support glycemic regulation by providing measurement data on patient status. Each sensor coupled to processor 270 can provide different analog waveforms that may require translation when converted to digital. In general, sensor interface, 298, analog signal processing 296, and ADC 278 buffers, translates, and converts an analog signal output by a sensor to digital that is received by processor 270. Sensors are typically limited in their ability to drive a load. For example, the glucose sensor can be coupled to sensor interface 298. The glucose sensor disclosed herein outputs a current signal. Sensor interface 298 provides an interface that couples to the glucose sensor without degrading the current signal output by the glucose sensor for measurement. The input of sensor interface 298 can have high resistance and low capacitance to lightly load an output of a sensor. In one embodiment, the current signal of the glucose sensor can be translated to a voltage signal. Sensor interface 298 can provide a signal buffer that can drive loads at levels typically required by circuitry that a sensor by itself cannot drive. Analog signal processing 296 couples to the output of sensor interface 298 and provides a translated signal. The translated signal output by sensor interface 298 is of a form that can then be received by ADC 278 for conversion to a digital format that is received by processor 270.

In one embodiment, processor 270 couples to digital logic 274. Digital logic 274 is configured to provide an interface to drive circuitry and further control outputs such as status outputs 282, E-stim outputs 284, US-stim outputs 286, and pump outputs 288. Digital logic 274 can comprise a programmable logic array (PLA). Analog circuitry can be coupled to digital logic 274 if required. Status outputs 282 can be one or more outputs for providing status of the artificial pancreas system. Status outputs 282 can couple to visual, audible, or haptic devices for providing different status indicators of the artificial pancreas system. E-stim outputs 284 corresponds to a signal for electrical stimulation. Referring briefly to FIG. 8, E-stim outputs 284 couples electrodes 228 and electrode 230 of insulin delivery system 204. E-stim outputs 284 flank the outlet port 226 for providing localized electrical stimulation to tissue in proximity to outlet port 226. E-stim outputs 284 couple to tissue adjacent to the artificial pancreas system to support opening channels and driving large molecules such as insulin closer to the venous system for absorption before breaking down. The electrical field or electrical stimulation created by E-stim outputs 284 changes cell membrane properties and can also support movement of ions in saliva or GIT fluid. In one embodiment, the electrical stimulation is an alternating electric field that stimulates tissue local to the port of the artificial pancreas system that is delivering insulin. In one embodiment, the artificial pancreas system can move or reposition within the mouth naturally. The movement of the artificial pancreas system relocates the port delivering insulin and thereby E-stim outputs 284. Thus, the tissue being stimulated is not static but moves as the artificial pancreas system moves naturally within the mouth. In other words, electrical stimulation does not occur on the same tissue all the time. Alternatively, the artificial pancreas system can be moved or adjusted by the patient to relocate the device within the mouth to prevent electrical stimulation in the same spot over a long period of time.

Pump outputs 288 drive pump 240 of FIGS. 9 and 10. In one embodiment, pump outputs 288 have a delivery cycle and fill cycle. Pump outputs 288 drive pump with pulses to support delivery of medicine or insulin in nano-liter quantities. In one embodiment, the voltage used to operate pump 240 can also be used to deliver electrical stimulation. In one embodiment, pump outputs 288 can be transmitted to an external pump to control the delivery and fill cycles. US-stim outputs 286 comprises an ultrasonic signal that is configured to provide ultrasound stimulus to the tissue adjacent to the artificial pancreas system to support exposure of the venous system to promote absorption of a medicine such as insulin. In one embodiment, US-stim outputs 286 couple to the pump of the artificial pancreas system to modulate the pump output with an ultrasonic frequency. The pump disclosed herein can be pump 240 shown in FIG. 9 and FIG. 10 of artificial pancreas system 200. In one embodiment, the pump can deliver insulin during a pump output cycle corresponding to a first frequency. During at least a portion of the pump delivery cycle, the pump can be modulated at a second frequency where the second frequency is greater than the first frequency. For example, the pump receives pump outputs 288 that generate a delivery cycle and a fill cycle of pump 240. For example, the delivery and fill cycle of pump 240 can operate pump 240 at a frequency such as 125 hertz. In one embodiment, the amount of medicine delivered can be measured in nano-liter quantities per delivery cycle. US-stim outputs 286 can be operated at frequencies substantially greater than 125 hertz such as the ultrasonic range. The medicine, insulin, or saliva is pulsed or vibrated at ultrasonic frequencies such as 1.5 megahertz by pump 240 to support placement of large molecules such as insulin into channels formed in the tissue nearer the venous system for more rapid absorption. The channels in the tissue adjacent to the artificial pancreas system are formed by ultrasonic fluid movement, electrical stimulation, or a combination of both. In the example, the pump can be a diaphragm pump with the diaphragm being modulated with the ultrasonic frequency such that the fluid delivered by pump 240 is pulsed or vibrated at or near the pump outlet near the buccal tissue of the mouth. Alternatively, the pump can include a second diaphragm that can be operated at ultrasonic frequencies. The first diaphragm of the pump can be operated at the first frequency to deliver a predetermined volume of insulin. The second diaphragm can be operated at ultrasonic frequencies to ultrasonically pulse the insulin and fluid to support channel opening in the tissue adjacent to the port of the pump to move the insulin closer to the venous system. In on embodiment, the second diaphragm is aligned to pulse the insulin and fluid in a similar direction as the first diaphragm.

FIG. 12 is an illustration of pump 240 in accordance with an exemplary embodiment. Pump 240 is configured to be used in artificial pancreas system 200 that can be placed in the mouth as shown in FIGS. 8-10. In one embodiment, pump 240 is a diaphragm pump having a small form factor to fit in the mouth or GIT. In one embodiment, pump 240 can be a MEMs diaphragm or operated piezo-electrically. Pump 240 includes a housing 318 having an inlet port 310 configured to pump in a fluid within a chamber 308 and an outlet port 226 configured to pump out the fluid within chamber 308. In one embodiment, pump 240 does not empty chamber 308 when pumping out fluid at outlet 312 but delivers a portion of the volume within chamber 308. In one embodiment, pump 240 further comprises a diaphragm 302, a piezo-electric membrane 304, an inlet flap 314, and an outlet flap 316. Diaphragm 302 couples to housing 318 and forms a boundary wall for pump chamber 308. In one embodiment, diaphragm 302 comprises a flexible material. Piezo-electric membrane 304 couples to diaphragm 302 to produce movement under electrical stimulation. In one embodiment, piezo-electric membrane 302 couples to pump outputs 288 of FIG. 11. The electric signal of pump outputs 288 produces a deflection in piezo-electric membrane 304. In one embodiment, piezo-electric membrane 304 can flex diaphragm 302 inward and outward where inward flexing of diaphragm 302 reduces the volume within chamber 308 and outward flexing of diaphragm 302 increases the volume within chamber 308. Inlet port 310 couples to a supply of medicine. In the example, the medicine is insulin. An outward flexing of diaphragm 302 by piezo-electric membrane 304 increases the volume within chamber 308 causing inlet flap 314 to open allowing insulin to enter chamber 308 from inlet port 310. The increase in volume within chamber 308 causes outlet flap 316 to close such that no insulin leaves at outlet port 226. This is called a fill cycle of pump 240. Conversely, an inward flexing of diaphragm 302 by piezo-electric membrane 304 decreases the volume within chamber 308 causing inlet flap 314 to close such that no insulin enters chamber 308 from inlet port 310. The decrease in volume within chamber 308 causes outlet flap 316 to open whereby insulin is delivered at output port 226. This is called the delivery cycle of pump 240. Thus, pump 240 when operated for medicine delivery has a repeating fill cycle and delivery cycle until a sufficient quantity of medicine has been delivered. In one embodiment, the amount of insulin delivered corresponds to the difference in volume of chamber 308 when diaphragm 308 is not flexed to the amount displaced when diaphragm 302 is flexed inward. In one embodiment, the fill and delivery cycle is performed at 125 hertz and nano-liter quantities of insulin is output during the delivery cycle. This is similar to how a pancreas delivers small quantities of insulin in bursts.

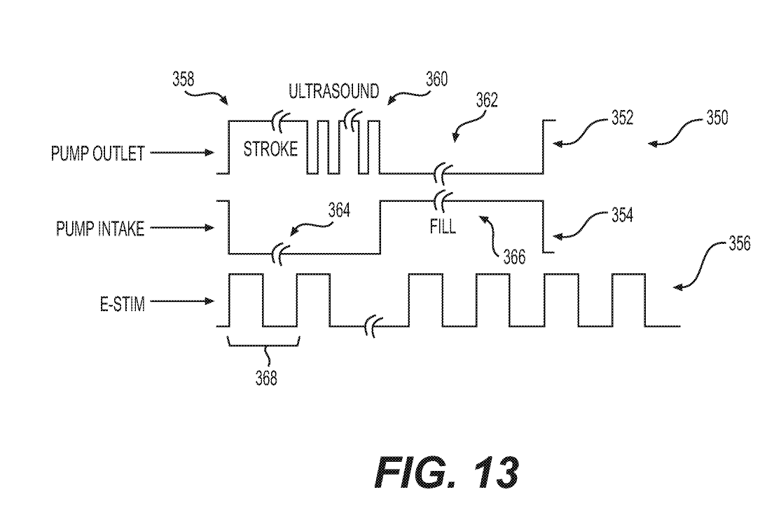

FIG. 13 is an illustration of a timing diagram 350 of pump 240 in accordance with an example embodiment. Applicant will refer to components of FIG. 12 to describe timing diagram 350. Timing diagram 350 discloses a diagram 352, a diagram 354, and a diagram 356. Diagram 352 illustrates when medicine or insulin is delivered from chamber 308 at output port 226 of pump 240. Diagram 354 illustrates when medicine or insulin is received within chamber 308 through inlet port 310 of pump 240. Diagram 356 corresponds to when electrical stimulation is provided to tissue in proximity to outlet port 226 by electrodes 228 and 230 shown in FIG. 8. In one embodiment, pump 240 delivers insulin at output port 226 corresponding to pulse 358 of diagram 352 reducing volume of chamber 308. In one embodiment, diaphragm 302 is modulated with an ultrasound signal indicated by pulses 360 having a much shorter pulse width than pulse 358. In one embodiment, pulse 358 pushes the insulin out of chamber 308 of pump 240 followed by high frequency pulses 360 output by diaphragm 302 that disrupt the tissue in proximity to the output port 226 to create channels in the tissue whereby the insulin can be delivered closer to the underlying capillary system for faster absorption into the venous system. Although pulse 358 and pulses 360 are shown having similar magnitudes they can be different or adjusted. A period 362 where no insulin is delivered at output port 226 follows pulses 360. Alternatively, pulses 360 can be modulated on diaphragm 302 during the entirety of pulse 358.

Diagram 354 illustrates action at inlet port 310. A delivery cycle occurs during pulse 358 and pulses 360 such that inlet flap 314 is closed, outlet flap 316 is open. No medicine or insulin is provided into chamber 308 via inlet port 310 during the delivery cycle. The fill cycle occurs after the delivery cycle ends. Piezo-electric membrane 304 flexes diaphragm 302 closing outlet flap 316 and opens inlet flap 310 by increasing the volume of chamber 308. Medicine or insulin coupled to inlet port 310 is coupled past inlet flap 314 to fill chamber 308. The pattern of insulin delivery and filling chamber 308 of pump 240 can be repeated as many times as necessary to deliver a predetermined amount. Diagram 356 corresponds to electrical stimulation provide by electrodes 228 and 230 to tissue in proximity to outlet port 226 of FIG. 8. Electrical pulses that generate non-static electrical fields are provided to the tissue during both the delivery and fill cycles to support tissue disruption and the formation channels for insulin delivery. In one embodiment, E-stim 284 of FIG. 11 can have a cycle of 250 hertz. The magnitude of E-stim 284 and the cycle that it is delivered can be varied under control of processor 270 or digital logic 274. Alternatively, the electrical pulses can be turned off during a portion of the fill cycle.

In one embodiment, piezo-electric membrane 304 cannot operate at ultrasonic frequencies. In other words, applying ultrasonic stimulus to piezo-electric membrane 304 may not generate pulses or vibration of the diaphragm 302 of sufficient magnitude to generate channels or disrupt tissue to enhance insulin delivery. A piezo-electric membrane 306 may be used to provide the ultrasonic pulsing of the insulin as it is delivered. Piezo-electric membrane 306 can couple to US-stim outputs 286 of FIG. 11. In one embodiment, piezo-electric membrane 306 can be mounted adjacent to piezo-electric membrane 304 to flex diaphragm 302. Alternatively, piezo-electric membrane 306 can be mounted on top of piezo-electric membrane 304 such that the ultrasound pulses couple through piezo-electric membrane 304 to diaphragm 302. In general, the ultrasonic pulsing of diaphragm 302 pulses the insulin as it is delivered out of outlet port 226. Thus, the ultrasonic pulsing is local to where the insulin is delivered to the tissue.

In one embodiment, the ultrasonic pulsing is used to create cavitation in proximity to the tissue where the medicine or insulin is being pumped for absorption. Pulsing or vibrating to move a liquid at ultrasonic frequencies can create voids related to the force acting on the liquid. The voids have no liquid in them. In the example, pump 240 is pumping medicine or insulin near tissue of the mouth or tissue within the GIT. There will also be saliva or GIT fluid respectively in the mouth or the GIT. In general, the voids are formed by rapid changes of pressure in the liquid where the pressure is low. Implosion of the voids can create shock waves that can be used to disrupt the tissue in the area of the cavitation to form channels or openings. Pump 240 can deposit medicine or insulin into the channels or openings formed by cavitation where large molecules such as insulin can enter. The channels place the insulin close to the capillary system beneath the tissue where it can be absorbed and delivered by the venous system.

In one embodiment, pump 240 pumps fluid adjacent to tissue and near outlet port 226 to support large molecule transport past buccal tissue near capillaries for absorption. In one embodiment, pump 240 delivers medicine at a rate of 125 hertz. In one embodiment, piezo-electric membrane 304 modulates diaphragm 302 with a 1.5 megahertz signal. In one embodiment, the 1.5 megahertz modulation occurs on the back half of the 125 hertz delivery cycle of pump 240. In one embodiment, the ultrasonic movement of the fluid occurs proximity to output port 226 of pump 240. This can produce local cavitation due to the excitation of the fluid, medicine, or insulin near output port 226 or tissue adjacent to output port 226. In general, the ultrasonic excitation occurs where medicine or insulin is being targeted. In one embodiment, output port 226 is about 2 millimeters in diameter. In one embodiment, the delivery of medicine or insulin comprises more than 20 delivery cycles each having ultrasound pulses super imposed on the last half of the delivery cycle. In one embodiment, the repetition rate of 20 or more delivery cycles can be repeated approximately every 30 seconds or as slow as several minutes until the appropriate amount of medicine or insulin has been delivered. This type of delivery of small quantities of insulin delivered over an extended time period is similar to how a pancreas delivers insulin. It should be noted that a signal at pump outputs 288 of FIG. 11 coupled to pump 240 of FIG. 12 can be varied in magnitude and cycle time to control how much medicine or insulin is delivered and at what rate the medicine or insulin is delivered. Similarly, US-stim outputs 286 can also be varied in magnitude and cycle time to control movement of the medicine or insulin and an amount of cavitation that occurs. In one embodiment, ultrasound excitation of the fluid or medicine and electric field modulation of the tissue in proximity to port 226 of pump 240 can occur at the same time such that pump 240 is driven and then extends for some time after the pump delivery cycle ceases, to aid in further absorption of the dispensed medicine or insulin. The ultrasonic or electric field burst width is variable and can be adjusted to help calibrate closed loop glycemic control. The burst repetition rate of US-stim 286 and E-stim 284 can be controlled by an algorithm that modulates to maintain glycemic management once the glucose sensor and pump characteristics are calibrated.

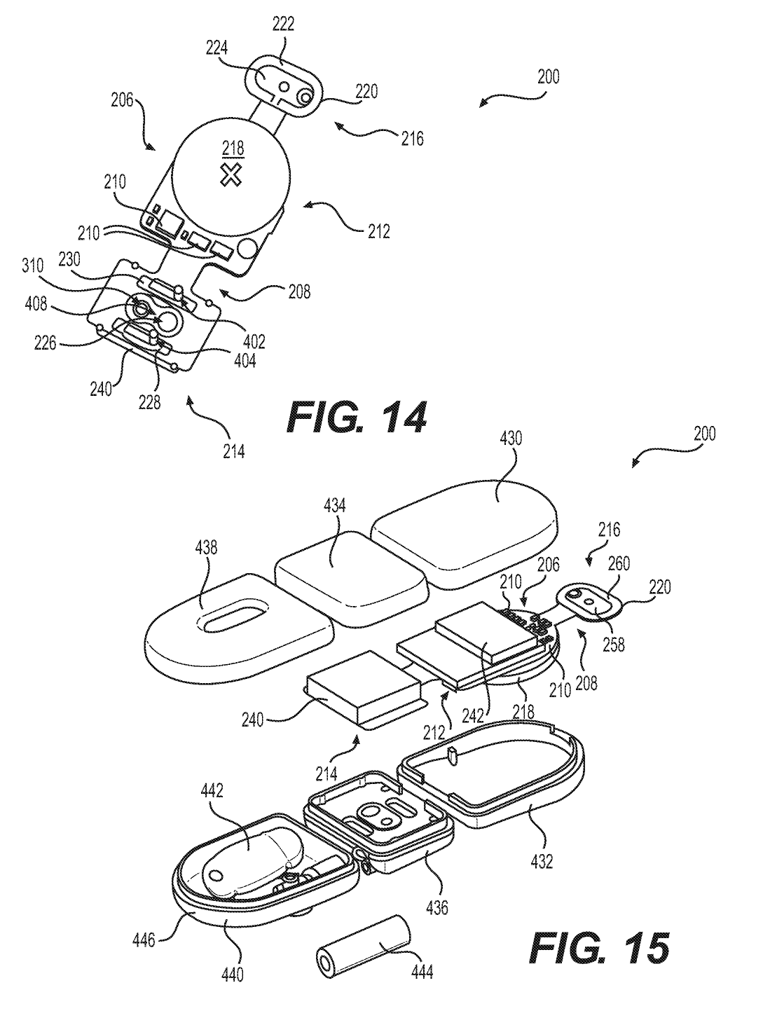

FIG. 14 is an illustration of artificial pancreas system 200 in accordance with an example embodiment. In one embodiment, artificial pancreas system 200 is configured to displace less than 1 cubic centimeter in volume and have a thickness of less than 4 millimeters. Moreover, artificial pancreas system 200 is flexible to allow bending to fit in areas of the mouth or GIT. For example, between the cheek and gum of the mouth. In one embodiment, a button can be cemented to a molar of the mouth. Artificial pancreas system 200 can be tethered or snapped to the button to retain it between the cheek and gum or underlying the tongue. As mentioned previously, artificial pancreas system 200 is placed in the mouth non-invasively for greater than one hour and typically between 3 days to two weeks configured to measure glucose and deliver insulin. Artificial pancreas system 200 can also have sensors listed herein above to measure patient status and activity to further refine insulin delivery. Artificial pancreas system 200 comprises a first section 212, a second section 216, and a third section 214. First section 212 comprises electronic circuitry 206, and a power source 218. Second section 216 comprises glucose sensor 220 configured to measure glucose levels in saliva of the mouth or fluid in the GIT. Third section 214 comprises a pump configured to pump medicine or insulin.

Inlet port 310 of pump 240 is configured to receive the medicine or insulin. Outlet port 226 of pump 240 is configured to pump out the medicine or insulin. A gasket 408 couples to inlet port 310 and outlet port 226 of pump 240. Gasket 408 includes openings for inlet port 310 and outlet port 226. A stim lead 402 extends from electrode 230. Similarly, a stim lead 404 extends from electrode 228. Stim leads 402 and 404 are configured to couple to tissue adjacent to artificial pancreas system 200 when placed in the mouth or GIT. Stim leads 402 and 404 provide a varying electric field to the tissue to support absorption of the medicine or insulin delivered by pump 240. In one embodiment, stim leads 402 and 404 stimulate tissue with electric fields. In one embodiment, electric field stimulation of tissue with ultrasonic excitation of the fluid, medicine, or insulin being delivered can open up channels in mucous tissue. In one embodiment, cavitation due to ultrasound fluid excitation with electrical stimulation opens up tissue or disrupts tissue in proximity to outlet port 226 to electrochemically receive large molecules and place them closer to a venous system for more rapid absorption.

FIG. 15 is an illustration of an enclosure that protects artificial pancreas system 200 from an external environment in accordance with an example embodiment. In the example, three enclosures are used to house artificial pancreas system 200 for use in the mouth. As mentioned, artificial pancreas system 200 can be placed between the cheek and gums of the mouth or under the tongue. Alternatively, artificial pancreas system 200 can be protected by an overlying coating or over-molded to form an enclosure. In one embodiment, glucose sensor 220 is not placed in the housing and is exposed to saliva in the mouth. A first housing comprises a structure 430 and a structure 432 that are coupled together to house first section 212 of printed circuit board 208. Structures 430 and 432 enclose electronic circuitry 206, power source 218, processor 242, and electronic components 210. In one embodiment, structures 430 and 432 are hermetically sealed to prevent the ingress of particles or fluids. In general, the housings disclosed herein can be sealed by gaskets, adhesives, sealant, welding, and other materials or structures for hermeticity. A second housing comprises a structure 434 and a structure 436 that are coupled together to house the third section 214 of printed circuit board 208. Structures 434 and 436 enclose pump 240 and are hermetically sealed. Alternatively, pump 240 can be a sealed design and may not require structures 434 and 436. A third housing comprises structure 438 and structure 440 that are coupled together to enclose a bladder for storing medicine or insulin. The third housing can include a bladder 442, a valve 446, and one or more couplings. An external tube 444 is configured to couple bladder 442 to pump 240. In one embodiment, the first housing, the second housing, and the third housing can be overmolded to minimize form factor or comprise a urethane, polycarbonate or other biocompatible material.

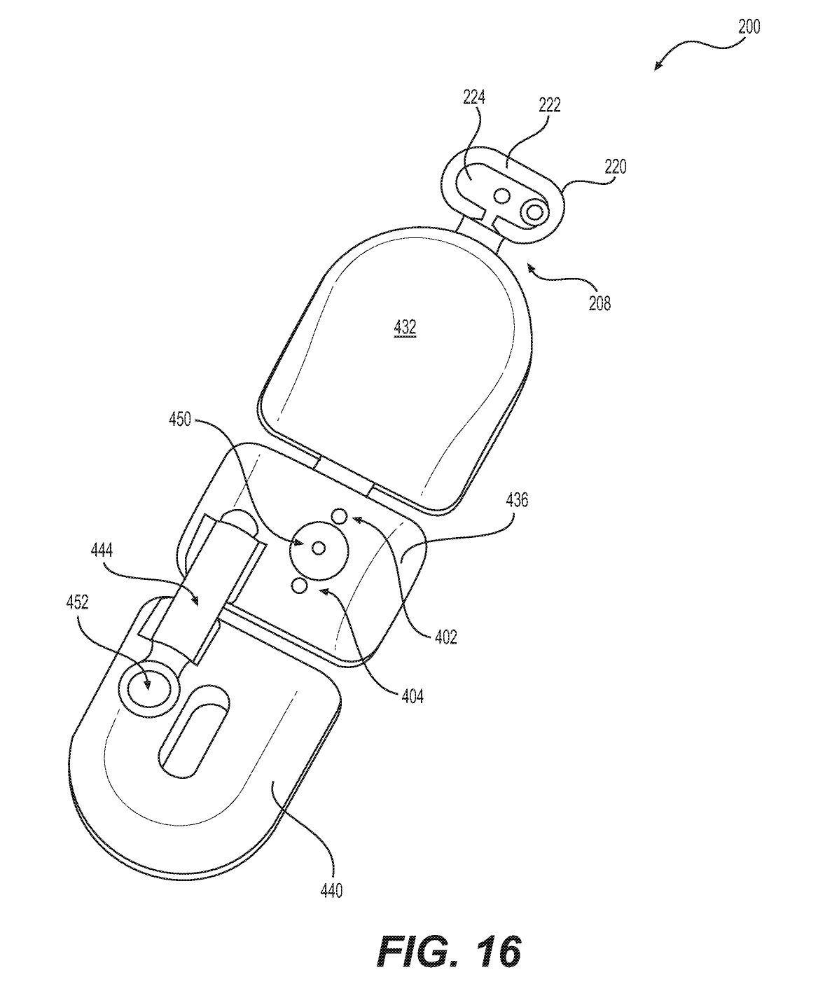

FIG. 16 is an illustration of artificial pancreas system 200 protected from an external environment in accordance with an example embodiment. Glucose sensor 220 is exposed for being immersed in saliva of the mouth or fluids in the GIT. Structure 432 of the first housing encloses the power source and electronic circuit of artificial pancreas system 200. Structure 436 of the second housing encloses the pump of artificial pancreas system 200. A discharge port 450, electrode 402, and electrode 404 couple through a surface of structure 436. Discharge port 450 is a funnel structure that extends from the surface opening into the interior of the second housing that couples to output port 226 of pump 240 as shown in FIG. 14. The bottom surface of the funnel structure couples to gasket 408 shown in FIG. 14. Stim leads 402 and 404 respectively extend from electrodes 230 and 228 (FIG. 14) through openings in the surface of structure 436. Stim leads 402 and 404 can be sealed to their respective openings to maintain hermeticity. Structure 440 of the third housing encloses the bladder for storing medicine or insulin. Structure 440 includes a fill port 452 that couples to the bladder within the third housing. Fill port 452 can be used to fill the bladder with medicine or insulin. Tube 444 is an external tube that couples to inlet port 310 of pump 240 of FIG. 14 and to the bladder. Thus, pump 240 receives medicine or insulin from the bladder. A pressure equalization valve can also couple to the bladder to equalize pressure as the bladder is drained.

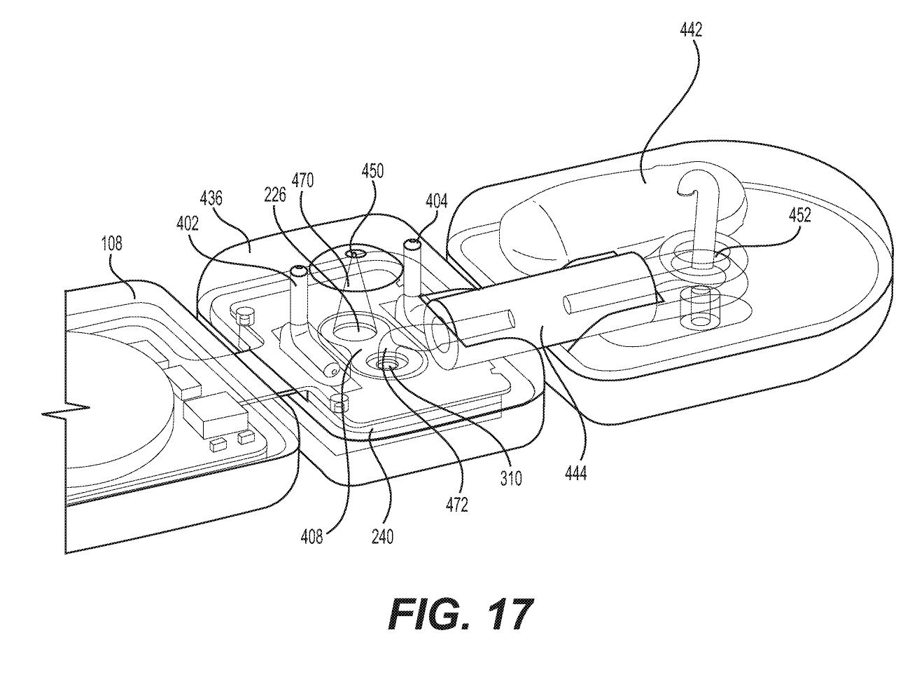

FIG. 17 is an illustration of bladder 442 coupling to pump 240 in accordance with an example embodiment. Fill port 452 couples to bladder 442. In one embodiment, medicine or insulin couples through a valve to fill bladder 442. Bladder 442 also couples to tube 444. Tube 444 is an external pipe that couples through a fitting to bladder 442. In one embodiment, tube 444 couples to fill port 452. Fill port 452 includes a valve that blocks tube 444 when medicine is delivered to bladder 442 through fill port 452. Conversely, the valve couples tube 444 to bladder 442 when fill port 452 is not being used. Tube 444 further couples to a fitting 472 that couples to inlet port 310 of pump 240. Medicine or insulin couples from bladder 442, through tube 444 and fitting 472 into inlet port 310 of pump 240 during a fill cycle. In one embodiment, fitting 472 couples to the gasket 408 to prevent leakage. In one embodiment, bladder 442 can use a pressure equalization cage around bladder 442 to prevent squeezing or deformation.

Discharge port 450 is an opening in funnel structure 470 on the surface of structure 436. Funnel structure 470 extends from the surface of structure 436 to couple to gasket 408 thereby sealing funnel structure 470 around outlet port 226 of pump 240. During a delivery cycle of pump 240, medicine or insulin is output from pump 240 into funnel structure 470 where the medicine or insulin is delivered to tissue adjacent to discharge port 450 when placed in the mouth. Stim electrodes 402 and 404 are shown extending through openings in the surface of structure 436 to touch tissue when placed in the mouth. Stim electrodes 402 and 404 apply an electrical field to the tissue to support the ingress of medicine or insulin past the tissue near capillaries of the venous system for absorption and delivery to the liver.

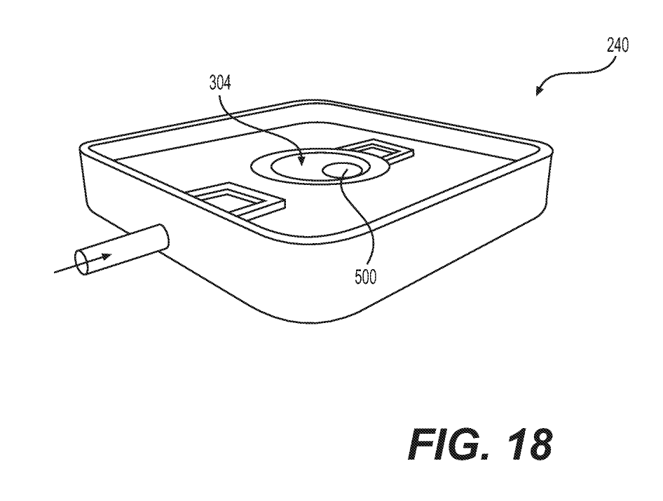

FIG. 18 is an illustration of a piezo-electric membrane 500 on a piezo-electric membrane 304 in accordance with an example embodiment. Piezo-electric membrane 304 couples to diaphragm 302. As mentioned previously, it may not be possible to produce ultrasonic pulsing from diaphragm 302. As shown in FIG. 12, a piezo-electric membrane 306 capable of ultrasonic pulsing can be placed adjacent to diaphragm 302 to excite diaphragm 302 to ultrasonically excite medicine or insulin delivered by pump 240. In one embodiment, piezo-electric membrane 500 can be coupled to piezo-electric membrane 304. Piezo-electric membrane 500 is capable of ultrasonic pulsing or vibration. Piezo-electric membrane 500 can ultrasonically excite medicine or insulin delivered by pump 240 through piezo-electric membrane 304 to diaphragm 302. In one embodiment, piezo-electric membrane can be formed together or bonded together.

While the present invention has been described with reference to particular embodiments, those skilled in the art will recognize that many changes may be made thereto without departing from the spirit and scope of the present invention. Each of these embodiments and obvious variations thereof is contemplated as falling within the spirit and scope of the invention.

1. An artificial pancreas system comprising:
electronic circuitry;
a glucose sensor coupled to the electronic circuitry; and
an insulin delivery system wherein the insulin delivery system is configured to couple to the electronic circuitry, wherein the electronic circuitry is configured to periodically or continuously measure glucose levels in saliva of the mouth, wherein the insulin delivery system is configured to deliver insulin to tissue in the mouth based on glucose measurement data from the glucose sensor, and wherein the artificial pancreas system is configured to electrically stimulate the tissue in the mouth to support delivery of the insulin below a tissue surface.

2. The system of claim 1 wherein the artificial pancreas system is configured to form channels in the tissue and wherein a portion of the delivered insulin is within the channels in the tissue to be absorbed by the venous system of the mouth.

3. The system of claim 1 wherein the artificial pancreas system is configured to be in the mouth greater than one hour.

4. The system of claim 1 wherein the artificial pancreas system further includes a second glucose sensor.

5. The system of claim 1 further including a first stim lead and a second stim lead configured to couple to the tissue of the mouth.

6. The system of claim 1 wherein the insulin delivery system includes a diaphragm pump coupled to an insulin reservoir, wherein the diaphragm pump is configured to operate at a first frequency comprising a fill cycle and a delivery cycle, and wherein a diaphragm of the diaphragm pump is modulated at a second frequency.

7. The system of claim 6 wherein the second frequency is ultrasonic.

8. The system of claim 6 wherein the insulin delivery system includes a discharge port and wherein the insulin delivery system is configured to produce cavitation in the mouth.

9. An artificial pancreas system comprising:
electronic circuitry;
a glucose sensor coupled to the electronic circuitry; and
an insulin delivery system including a diaphragm pump wherein the insulin delivery system is configured to couple to the electronic circuitry, wherein the electronic circuitry is configured to periodically or continuously measure glucose levels in saliva of the mouth, wherein the insulin delivery system is configured to deliver insulin to tissue in the mouth based on glucose measurement data from the glucose sensor, and the insulin delivery system is configured to excite the delivery of insulin ultrasonically.

10. The system of claim 9 wherein the insulin delivery system includes a diaphragm pump coupled to an insulin reservoir, wherein the diaphragm pump is configured to operate at a first frequency comprising a fill cycle and a delivery cycle, and wherein a diaphragm of the diaphragm pump is modulated at a second frequency.

11. The system of claim 10 wherein the second frequency is ultrasonic.

12. The system of claim 10 wherein the insulin delivery system is configured to produce cavitation in proximity to the tissue of the mouth.

13. The system of claim 9 wherein the artificial pancreas system is configured to form channels in the tissue and wherein a portion of the delivered insulin is within the channels in the tissue to be absorbed by the venous system of the mouth.

14. The system of claim 9 wherein the artificial pancreas system is configured to electrically stimulate the tissue in the mouth to support delivery of the insulin below a tissue surface.

15. The system of claim 14 further including a first stim lead and a second stim lead configured to couple to the tissue of the mouth.

16. The system of claim 9 wherein the artificial pancreas system further includes a second glucose sensor.

17. An artificial pancreas system comprising:
electronic circuitry;
a glucose sensor coupled to the electronic circuitry; and
an insulin delivery system including a diaphragm pump wherein the insulin delivery system is configured to couple to the electronic circuitry, wherein the electronic circuitry is configured to periodically or continuously measure glucose levels in saliva of the mouth, wherein the insulin delivery system is configured to deliver insulin to tissue in the mouth based on glucose measurement data from the glucose sensor, and the insulin delivery system is configured to produce cavitation in the mouth.

18. The system of claim 17 wherein the insulin delivery system is configured to excite the delivery of insulin ultrasonically.

19. The system of claim 17 wherein the insulin delivery system includes a diaphragm pump coupled to an insulin reservoir, wherein the diaphragm pump is configured to operate at a first frequency comprising a fill cycle and a delivery cycle, and wherein a diaphragm of the diaphragm pump is modulated at a second frequency.

20. The system of claim 17 wherein the artificial pancreas system is configured to form channels in the tissue and wherein a portion of the delivered insulin is within the channels in the tissue to be absorbed by the venous system of the mouth.

21. The system of claim 21 further including a first stim lead and a second stim lead configured to couple to the tissue of the mouth.
